(19) United States
(12) Patent Application Publication
(10) Pub. No.: US 2026/0263742 A1
(43) Pub. Date: Sep. 10, 2026

(54) GUIDEWIRE DIRECTIONAL POSITIONING AND RE-ENTRY CATHETER WITH ROTATIONAL INDICIA

(71) Applicant: Simpson Interventions, Inc., Campbell, CA (US)

(72) Inventors: John B. Simpson, Campbell, CA (US); Doug Rowe, Campbell, CA (US); August Pombo, Campbell, CA (US); Kin F. Chan, Campbell, CA (US); Thi Thanh Hue Ho, Campbell, CA (US)

(21) Appl. No.: 19/167,799

(22) PCT Filed: Mar. 21, 2024
(86) PCT No.: PCT/US2024/021005

§ 371 Date: Sep. 22, 2025

Related U.S. Application Data
(60) Provisional application No. 63/491,365, filed on Mar. 21, 2023.

(51) Int. Cl. A61M 25/00; A61B 17/00; A61B 17/22; A61B 90/00; A61M 25/01

(52) U.S. Cl. CPC A61M 25/0028; A61B 17/00234; A61B 17/22; A61B 90/39; A61M 25/0108; A61B 2017/00238; A61B 2017/00305; A61B 2017/22038; A61B 2017/22095; A61B 2090/3966; A61M 2025/0037; A61M 2205/32; A61M 2210/12

(57) ABSTRACT

A multi-lumen catheter to perform re-entry of a body lumen to bypass an intraluminal lesion includes rotational indicia to permit the determination of catheter and/or guidewire orientation via external imaging, such as a fluoroscopy system.

### BACKGROUND

This invention relates generally to minimally invasive vascular medicine, and more specifically to methods and apparatus for directional control of guidewire advancement/positioning and re-entry catheter within the vasculature as well as to bypass intraluminal obstructions, respectively.

### SUMMARY

Anatomically complex chronic total occlusions (CTO) often require leveraging the extraplaque or formerly “subintimal” space for successful CTO recanalization. Antegrade dissection and reentry (ADR) technique is an important part of the hybrid approach to contemporary CTO percutaneous coronary intervention (PCI). However, despite the availability of dedicated re-entry devices, the success rate for ADR is around 50-60% in large registries. This is often due to large extraplaque hematoma formation leading to loss of distal vessel visualization and loss of guidewire support and maneuverability making re-entry challenging. Both existing ADR and antegrade wire escalation are blinded strategies given the lack of realtime visual guidance by angiography and their inability to provide a high degree of precision in steering and advancing a re-entry device.

Embodiments are directed to methods and systems configured to perform fluoroscopic and/or intravascular image guidance and/or antegrade dissection and reentry for the treatment of chronic total occlusion of a blood vessel, including coronary and peripheral vasculature. The catheter may be configured to support and aim steerable guidewires with radiopaque indicia on the catheter to allow the user to confirm the rotational orientation of the catheter via fluoroscopy to facilitate the desired guidewire re-entry form the subintimal space back into the true lumen of a vessel. Similar radiopaque indicia may be used to direct guidewires from a vessel into a side branch that is typically not easily accessible such as those at oblique angle.

In one embodiment, a catheter is provided, comprising a tubular catheter body, the tubular catheter body comprising a first lumen comprising a first proximal opening, a first distal opening and a first straight lumen therebetween, a second lumen comprising second proximal opening, a second distal opening, a second straight lumen and a curved lumen, wherein the second distal opening is proximal to the first distal opening and located in a sidewall of the tubular catheter body, and wherein the curved lumen is located between the second straight lumen and the second distal opening, a radiopaque angled ramp structure, wherein the first straight lumen and second straight lumen are parallel and located in a first plane, wherein the second straight lumen and the curved lumen are located in a second plane that is non-parallel to the first plane, and wherein the radiopaque angled ramp structure extends from the first plane and is in alignment with the second plane. The radiopaque angled ramp structure may comprise an obtuse angled surface relative to the second straight lumen of the second lumen, the obtuse angled surface lying in a third plane. The radiopaque angled ramp structure may further comprise a distal end with an orthogonal configuration. The radiopaque angled ramp structure may further comprise a distal end with a 30 to 60 degree angled surface lying in a fourth plane. The radiopaque angled ramp structure may further comprise a notch located between the obtuse angled surface and the distal end. The notch may comprise an angled notch surface located in a fifth plane that is different from the third plane. The fifth plane may be axially rotated 45 degrees from the third plane. The fourth plane may be axially rotated 45 degrees from the fifth plane. The fourth plane may be axially rotated 90 degrees from the third plane. The catheter may further comprise a first circumferentially oriented arcuate radiopaque band spaced distally from the distal end of the ramp marker. The first circumferentially oriented arcuate radiopaque band may be a hemi-circumferential band. The catheter may further comprise a second circumferentially oriented arcuate radiopaque band spaced distally from the first circumferentially oriented arcuate radiopaque band, the second circumferentially oriented arcuate radiopaque band comprising a different rotational orientation than the first circumferentially oriented arcuate radiopaque band. The second circumferentially oriented arcuate radiopaque band may be a hemi-circumferential band. The second circumferentially second circumferentially oriented arcuate radiopaque band may be rotated 90 degrees from the first circumferentially oriented arcuate radiopaque band. The catheter of claim 3, further comprising a tubular radiopaque marker surrounding the first lumen and longitudinally aligned with the orthogonal configuration of the distal end of the ramp structure. The e angle between the first plane and second plane may be in the range of 60 to 80 degrees. The catheter may further comprise a base body coupled to the first lumen, the second lumen, and the angled ramp structure. The base body may comprise a base body flange extending from the base body, the flange comprising a radial longitudinal orientation and having a through hole. The angled ramp structures may comprise an elongate proximal tail, and an enlarged distal head. The enlarged distal head may comprise a tubular shape. The e enlarged distal head may further comprise a transverse opening. The second plane may be orthogonal or form an acute angle to the first plane.

### BRIEF DESCRIPTION OF THE DRAWINGS

FIG. 1A is a schematic representation of the coronary anatomy of a heart. FIG. 1B is a close-up schematic of the right coronary anatomy depicting the subintimal path of a re-entry guidewire.

FIG. 2A is a schematic transverse cross-sectional view of the coronary anatomy depicting various re-entry guidewire paths. FIG. 2B is a schematic longitudinal cross-sectional view of the coronary anatomy in FIG. 2A depicting various re-entry guidewire paths.

FIGS. 3A to 3C depict an exemplary fluoroscopy/cardiac catheterization lab with the fluoroscopy unit positions to perform AP, LAO and RAO imaging, respectively.

FIG. 4A is an isometric cutaway view of a distal region of an exemplary re-entry catheter with an angled notch. FIG. 4B is a schematic side view of the catheter in FIG. 4A.

FIGS. 5A to 5D are exemplary AP fluoroscopy images of the catheter in FIGS. 4A and 4B with the re-entry guidewire oriented at the 0, 90, 180 and 270 degree orientations toward the fluoroscope imager. FIGS. 5E and 5F are side and transverse schematic views of the catheter and re-entry guidewire in FIG. 5A. FIGS. 5G and 5H are side and transverse schematic views, respectively, of the catheter and re-entry guidewire in FIG. 5B. FIGS. 5I and 5J are side and transverse schematic views, respectively, of the catheter and re-entry guidewire in FIG. 5C. FIGS. 5K and 5L are side and transverse schematic views, respectively, of the catheter and re-entry guidewire in FIG. 5D.

FIG. 6 is an isometric cutaway view of a distal region of another exemplary catheter with multiple angled cuts.

FIGS. 7A to 7P are side and transverse schematic views, respectively of the catheter and re-entry guidewire in FIG. 6 at 0, 45, 90, 135, 180, 225, 270 and 315 degree orientations, respectively.

FIG. 8 is an isometric cutaway view of a distal region of another exemplary catheter with multiple rotational indicia.

FIGS. 9A to 9P are side and transverse schematic views, respectively of the catheter and re-entry guidewire in FIG. 8 at 0, 45, 90, 135, 180, 225, 270 and 315 degree orientations, respectively.

FIG. 10 is an isometric cutaway view of a distal region of another exemplary catheter with multiple rotational indicia.

FIGS. 11A to 11P are side and transverse schematic views, respectively of the catheter and re-entry guidewire in FIG. 10 at 0, 45, 90, 135, 180, 225, 270 and 315 degree orientations, respectively.

FIG. 12 is an isometric cutaway view of a distal region of another exemplary catheter with multiple rotational indicia.

FIGS. 13A to 13P are side and transverse schematic views, respectively of the catheter and re-entry guidewire in FIG. 12 at 0, 45, 90, 135, 180, 225, 270 and 315 degree orientations, respectively.

FIG. 14 is an isometric cutaway view of a distal region of another exemplary catheter with positioning lumen indicia bands.

FIGS. 15A to 15H are side and transverse schematic views, respectively of the catheter and re-entry guidewire in FIG. 14 at 270, 180, 90 and 0 degree orientations, respectively.

FIG. 16A is a side elevational view another exemplary catheter with rotational indicia. FIG. 16B is an isometric cutaway views of a distal region of the catheter in FIG. 16B.

FIGS. 17A and 17B are schematic transverse cross-sectional views depicting the ramp lumen rotational orientations in an orthogonal and acute angle positions.

### DETAILED DESCRIPTION

Embodiments are directed to systems, devices, and method for directional control of guidewire steering, advancement and positioning for utility in antegrade dissection and reentry. Such systems, devices, and methods may include an interventional re-entry catheters (“catheter”) having both flexibility to maneuver within the vasculature and torsional control to orientate the catheter, systems including the same, and method of using the same. An exemplary catheter includes a distal catheter region configured to be at least partially disposed in a body and a proximal catheter region configured to remain at least partially outside of the body. The distal catheter region includes a guidewire through lumen and a guidewire off-ramp lumen with a radio-opaque ramp indicator adjacent to the guidewire off-ramp lumen. The ramp indicator may comprise a non-orthogonal angled surface, and is located distal to the where the guidewire side lumen transitions from a longitudinal orientation to a side-angled orientation. The guidewire lumen is configured to receive a guidewire and includes a distal region and a proximal region. The proximal region of the guidewire through lumen and the proximal region of the guidewire off-ramp lumen may be parallel.

A guidewire inserted into the body of a patient, is then inserted into the through guidewire through lumen of the catheter to facilitate positioning of the catheter at a desired target location. Initially, antegrade wire escalation (AWE) techniques may be used in an attempt to cross a lesion to achieve true lumen-to-true lumen access. If the AWE technique fails, antegrade dissection and reentry is then attempted. A knuckle guidewire or other type of CTO crossing guidewire may be advanced to the lesion by a microcatheter and upon entry into the subintimal space, the microcatheter is replace with the reentry catheter to facilitate reentry back into the true lumen distal to the lesion.

A guidewire inserted into the body of a patient, is then inserted into the through guidewire through lumen of the catheter to facilitate positioning of the catheter at a desired target location. Often, antegrade wire escalation (AWE) techniques involve the deployment of a guiding catheter to provide structural support for the guidewire in the attempt to achieve true lumen-to-true lumen access. If the AWE technique fails, antegrade dissection and reentry is then attempted using either the same guiding catheter or by replacing it with a specialty re-entry microcatheter to attempt antegrade dissection and re-entry. At present, re-entry catheter lacks both visual guidance and torsional capability in directing and advancing the guidewire to objectively achieve successful re-entry. Most re-entry successes are chanced successes.

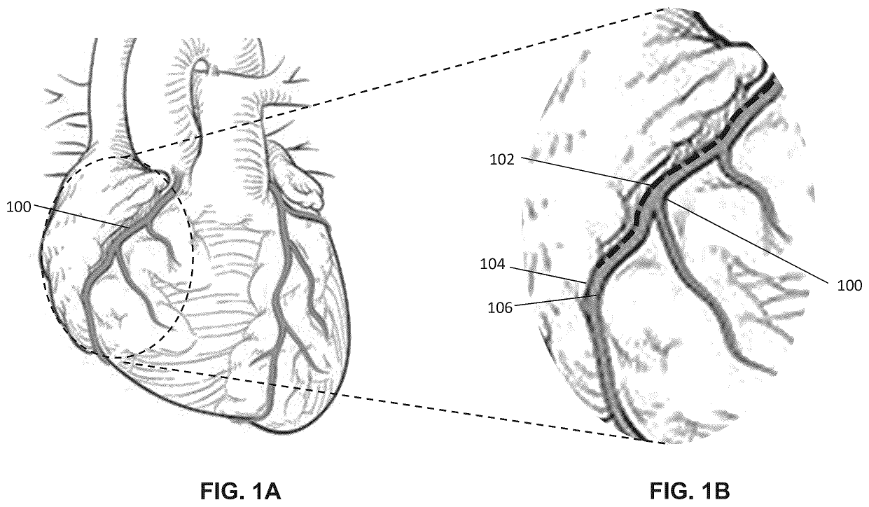

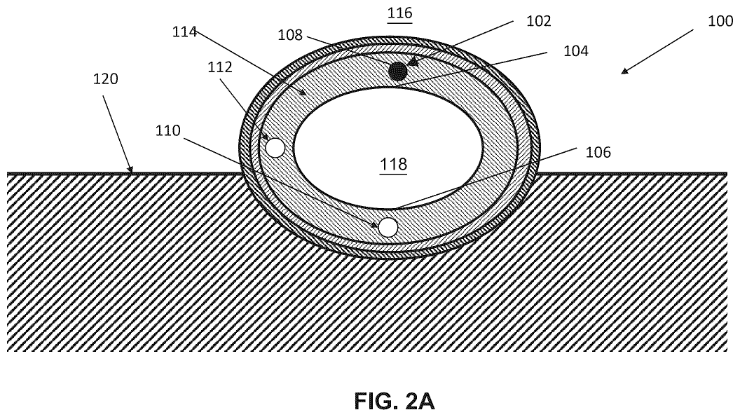

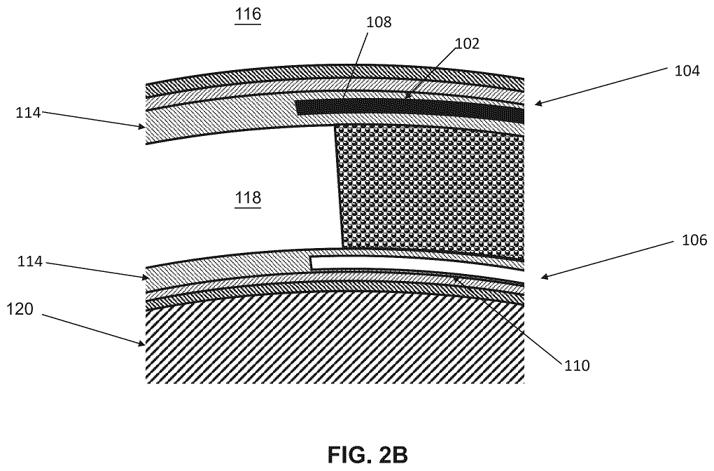

During use, it may be difficult to determine the orientation of the off-ramp relative to the body. However, referring to FIGS. 1A and 1B, it has been noted in clinical testing that devices that are deployed into the coronary arteries tend to flex outward and are biased against the greater curvature of the of the vessel 100. This is likely the result of the guidewire taking the configuration with the greatest stress relief. This is likely true whether the guidewire is deployed with or without a guiding or support catheter. A CTO crossing guidewire will thus typically enter the subintimal layer of a blood vessel 100 along a guidewire path 102 that tracks the greater curvature 104 of the vessel 100, compared to the lesser curvature 106. Referring to FIGS. 1B, 2A and 2B, the guidewire path 102 along the greater curvature 104 results in a lower stress in the guidewire 108. This is in contrast to a path 110, along the less curvature 106 or an intermediate path 112 intermediate to the greater and less curvatures, 104, 106, which would require increased stress and strain in the guidewire to achieve or maintain. Thus, the likely location of the guidewire 108 is between the pericardial space 116 and the true lumen 118 of the vessel 100, rather than between the true lumen 118 and the myocardium 120.

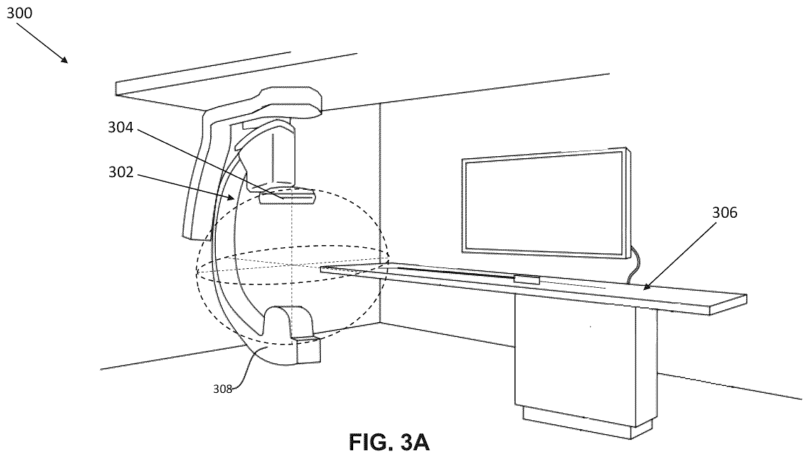

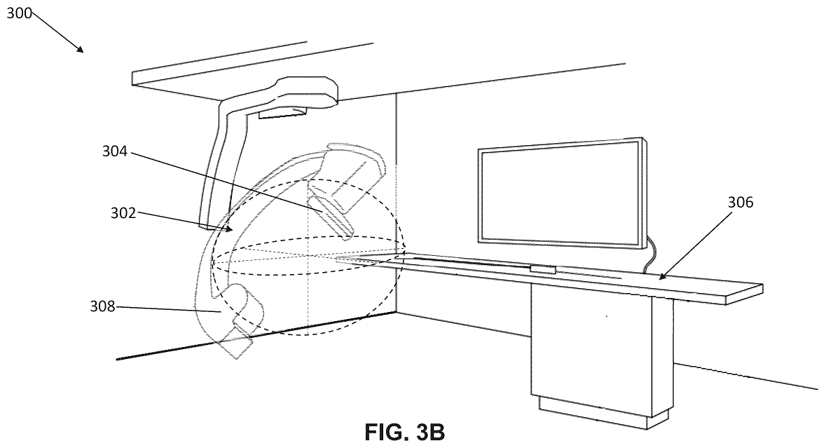

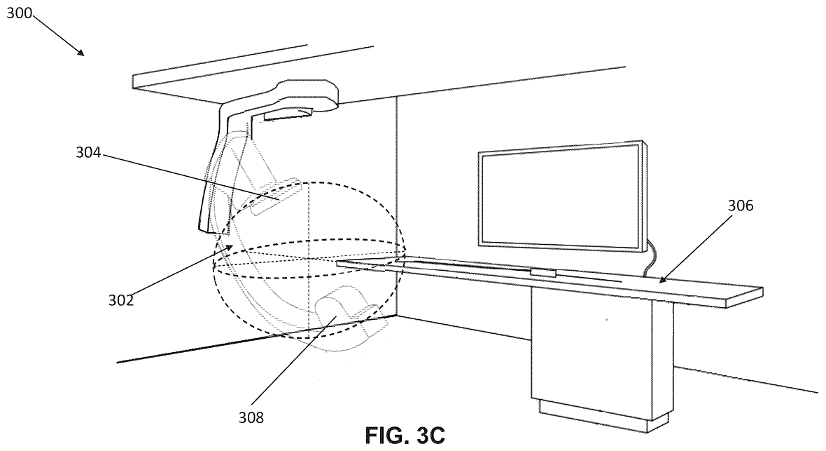

By taking advantage of this anatomical preference when a guidewire is crossing a CTO lesion, the angle of guidewire reentry from the subintimal region back into the true lumen may not require detailed imaging with intravascular ultrasound or optical coherence tomography for confirmation. Instead, with an understanding of the fluoroscopy imager imaging position, orientation of the reentry guidewire and catheter toward the center of the patient's cardiac anatomy may suffice for successful re-entry back into the true lumen. Thus, depending on the particular location of the lesion in the coronary anatomy, and the orientation of the fluoroscopy imager 300, e.g. in the anterior-posterior (AP) view (FIG. 3A), Left Anterior-Oblique (LAO) view (FIG. 3B), and Right-Anterior-Oblique (RAO) view (FIG. 3C), the likely relative re-entry guidewire angle can be determined. Although these views are standardized cardiac catheterization view and orientations, the angle of the C-arm 302 may be adjusted to maximize the structural separation between various catheter structures to increase the accuracy of catheter positioning. In the view depicted in FIG. 3A, the x-ray emitter 308 is below the plane of the procedure table 306 and the image intensifier/detector 304 above the plane of the table 306, without any cranial or caudal angulation. The LAO view depicted in FIG. 3B is typically used for imaging of the right coronary artery. In this orientation, the emitter and detector 308, 304 are rotated 60 degrees clockwise from the sagittal plane. The RAO view depicted in FIG. 3C is typically used for imaging of the left coronary vasculature, with the emitter and detector 308, 304 rotated 30 degrees counterclockwise from the sagittal plane. In each of the views, however, various adjustments to the rotation from the sagittal plane, or in the cranial or caudal angles, may be changed to optimize the fluoroscopic view of the catheter and/or anatomical structures.

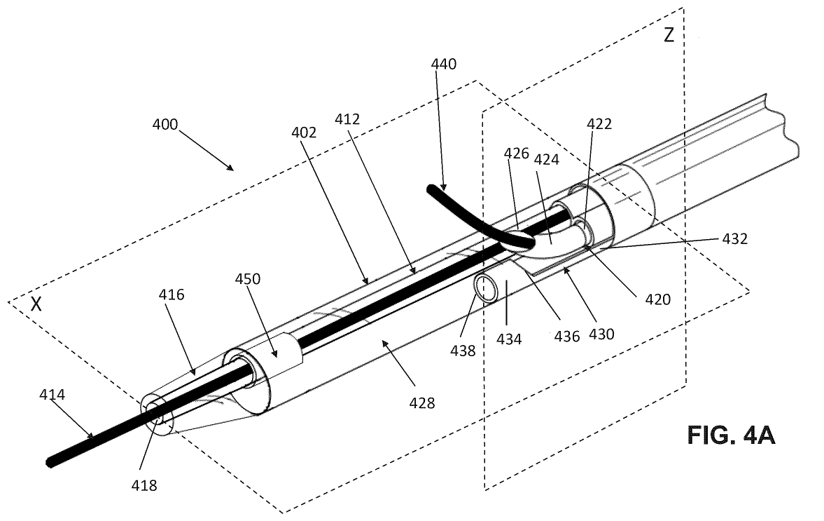

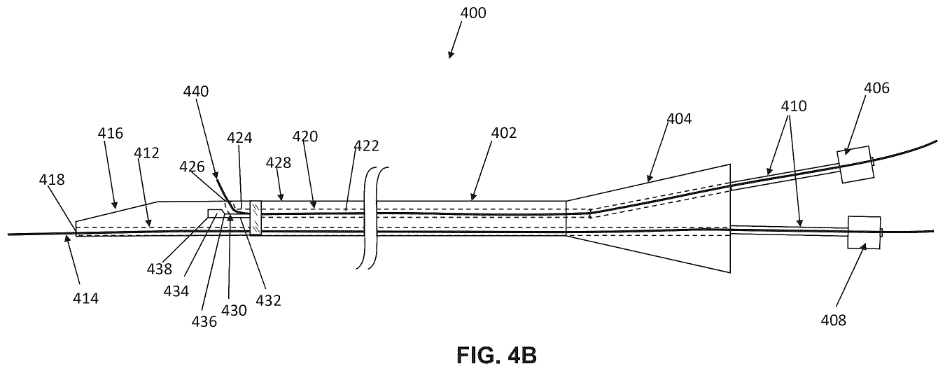

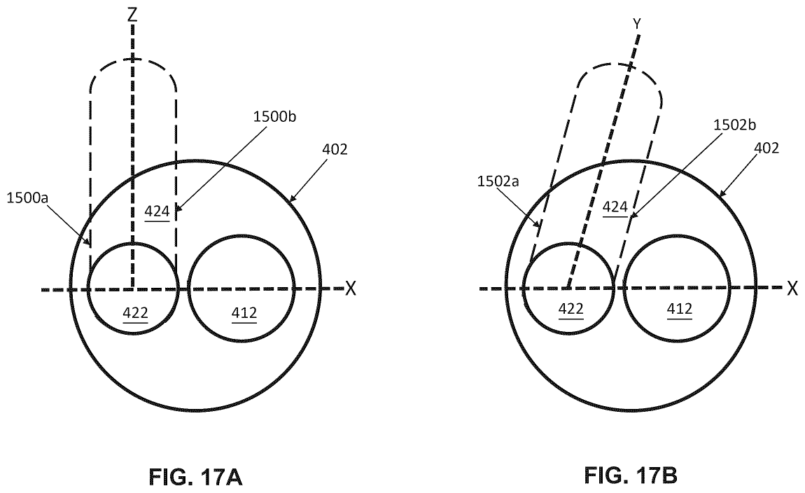

FIGS. 4A and 4B schematically depict one exemplary embodiment of the re-entry catheter. In FIG. 4B, the catheter 400 comprises a tubular body 402, proximal hub 404, and one or more ports 406, 408 attached to the hub 404 directly or via flexible tubing 410, as depicted in FIG. 4B. The tubular body 402 may comprise a first through lumen 412 into which a positioning guidewire 414 may be inserted and used to facilitate positioning of the catheter 400 during a procedure. The tubular body 402 may comprise a tapered distal region 416, wherein the distal opening 418 of the first through lumen 412 resides at its distal end. The tubular body 402 further comprises a second ramp lumen 420, which itself comprises a proximal linear segment 422 and a distal curved segment 424. The distal curved segment 424 has a distal opening 426 in the sidewall 428 of the tubular body 402 that is proximal to the distal opening 418 of the first through lumen 412. The second ramp lumen 420 may be used in conjunction with a reentry guidewire 440 to reenter the true lumen after the guidewire is located in the subintimal layer of the vessel. The tubular body 402 may also further comprise a ramp support or marker 430, which has a proximal segment 432 that comprises a co-axial hemi-tubular structure in which the proximal linear segment 422 of the ramp lumen 420 may partially reside or be otherwise located against. The ramp marker 430 further comprises a distal segment or head 434 that has an increased transverse dimension compared to the proximal linear segment 422, and an angled transition segment 436 therebetween. In this particular embodiment, the ramp marker 430 comprises a distal end 438 with a transverse cut, but in other variations, the distal end 438 may comprise a tapered or angled cut. And additional radiopaque marker band 450 may be provided elsewhere in the distal segment of the catheter 400, including around the first guidewire lumen 412 in the nosecone region 416 or in between the nosecone region 416 and the ramp marker 430, or within the tubular body 402 of the catheter spaced apart from the first guidewire lumen 412. Referring to FIG. 17A, in some variations, the first through lumen 412 and proximal linear segment 422 of the second ramp lumen 420 are located in the same first plane X, while the distal curved segment 424 and the proximal linear segment 422 also lie the same second plane Z that is orthogonal to the first plane X, as depicted in FIG. 17A. In other variations, as depicted in the exemplary variation in FIG. 17B, the proximal linear segment 422 and the distal curved segment 424 may lie in a non-orthogonal plane Y, at a non-orthogonal angle to plane X. In some variations, this planar angle may be in the range of 45 degrees to 135 degrees, or 70 degrees to 110 degrees, or 60 to 80 degrees, for example. For acute planar angles, the smallest angle may be limited by the size or diameter of the 1^{st} through lumen 412. A planar angle of less than 90 degrees may be beneficial in that the wall length with respect to plane Y is greater than the wall length with respect to plane Z.

For example, in FIG. 17A, the outer side wall 1500a is relatively short in Z plane, even relative to the opposing inner sidewall 1500b. Because of the shorter wall length, as the reentry guidewire is extended through the lumen 424, it may diverge more from the intended trajectory along the Z plane, due shorter runway length provided by the shorter wall lengths 1500A and 1500B. In contrast, FIG. 17B, the distal curved segment 424 has an acute angle of 70 degrees, to the X plane, but the outer side wall 1502a and the inner side wall 1502b are both longer along the Y plane than the outer and inner side walls 1500a, 1500b, and thus may exhibit less trajectory deviation from the Y plane, as a result of the greater support length or runway length of the distal curved segment 424 since is passes through more the tubular body 402 as a result of the change in angulation from an orthogonal orientation.

Referring back to the embodiment depicted in FIGS. 4A and 4B, the angled transition segment 436 of the ramp marker 430 may have a 135 degree angled surface as measured from the first plane and its proximal linear segment 422. Typically, the angle of the angled transition segment 436 will match the nominal exit angle formed by the proximal linear segment 422 and the distal curved segment 424, which may assist the user in predicted the exit trajectory of the reentry guidewire. In some other embodiments, the angled transition segment 436 and the exit angle may be in the range of 90 degrees to 170 degrees, 110 degrees to 150 degrees, 120 degrees to 150 degrees, or 135 degrees to 170 degrees. In some further variations, more obtuse angles may reduce divergence or variation from the predicted exit angle, as a result of the greater support length or runway length of the distal curved segment 424 through the tubular body 402 compared to relatively smaller angles. In addition, the distal segment 434 may comprise a tubular configuration with its increased transverse dimension and it angled transition segment 436 extending out of the Z plane, which allows it to be visible when the Z plane is aligned with the image intensifier of the fluoroscopy system.

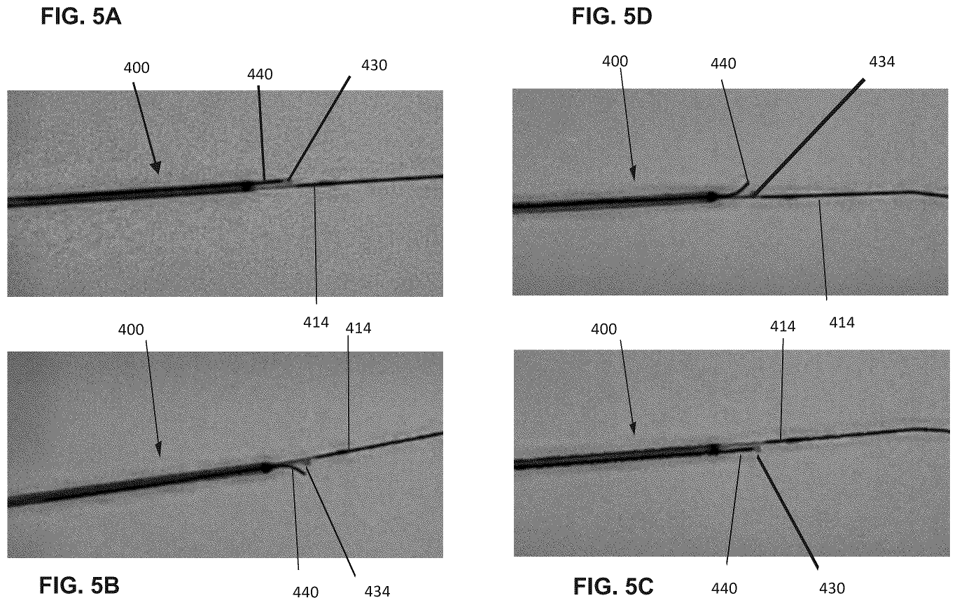

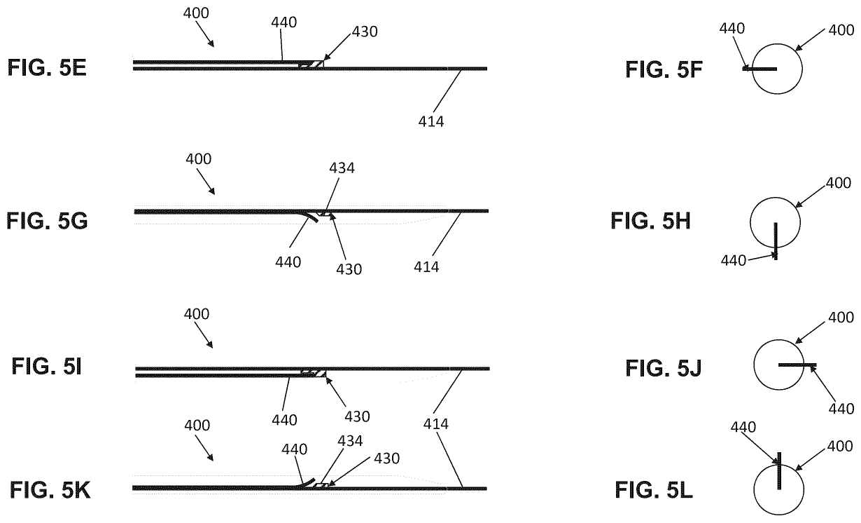

Referring now to FIGS. 5A to 5C, the appearance of the catheter 400 in the four orthogonal orientations relative to the orientation of the fluoroscopy arm is illustrated. In FIG. 5A, separation is seen between the positioning guidewire 414 extending out of the catheter 400 and the reentry guidewire 440, e.g. a “double” guidewire view. Since the reentry guidewire 440 and the ramp marker 430 located superiorly/cranially on the image, relative to the positioning guidewire 414, this corresponds with the distal opening of the second ramp lumen being oriented toward the image intensifier of the fluoroscopy system. This is also depicted schematically in FIGS. 5E and 5F. In axial cross sectional figures such as 5F, 5H, 5J and 5L, the 9 O'clock position is toward the image intensifier, while the 3 o'clock position is away from the image intensifier, and the 6 o'clock position is in the relative caudal direction relative to the image intensifier and the 12 o'clock position is cranial relative to the image intensifier.

In FIG. 5B, the catheter 400 has been rotated 90 degrees, such that the elongate structure of the positioning guidewire 414 is now overlapping or aligned with the reentry guidewire 440, e.g. a “single” guidewire view. Without extending the reentry guidewire 440 out of the distal opening of the second ramp lumen, it may be difficult to determine the whether the second ramp lumen is oriented to exit cranially or caudally. This is because the distal segment 434 of the ramp marker 430 is visualized extending caudally in FIG. 5B, it can be confirmed that the second ramp lumen 420 is oriented caudally, as depicted by the extended reentry guidewire 440. This is also depicted schematically in FIGS. 5G and 5H.

In FIG. 5C, the catheter 400 has been rotated another 90 degrees relative to its position in FIG. 5B. This is illustrated as the positioning guidewire 414 and the reentry guidewire 440 are one again separated, depicting two parallel guidewire lines, and no longer overlapping or aligned. In contrast to FIG. 5A however, the shorter reentry guidewire 440 is located caudally relative to the longer positioning guidewire 414, so that it is known to the operator that the second ramp lumen is oriented away from the image intensifier. This is also depicted schematically in FIGS. 51 and 5J.

In FIG. 5D, the catheter 400 has again been rotated approximately 90 degrees relative to the orientation depicted in FIG. 5C. The catheter 400 is back in a “single” guidewire view, but now the distal segment 434 of the ramp marker 430 is visualized extending cranially, thus indicating that the second ramp lumen 420 is oriented to point cranially, as confirmed by the extended reentry guidewire 440. This is also depicted schematically in FIGS. 5K and 5L.

The tubular body 402 may comprise a material with a high shear modulus and stiffness to provide high torquebility and pushability, kind resistance, with low friction, flexibility and trackability along the anatomy, such as HDPE, PEEK, or polyamide, and with optional coatings of PTFE or PU, for example. The tubular body 402 surface may additionally be partially or fully coated with hydrophilic coating to reduce friction and improve pushability. To improve the visibility of the ramp marker on fluoroscopy, the support may comprise stainless steel that is optionally coated with gold particles or be replaced with platinum.

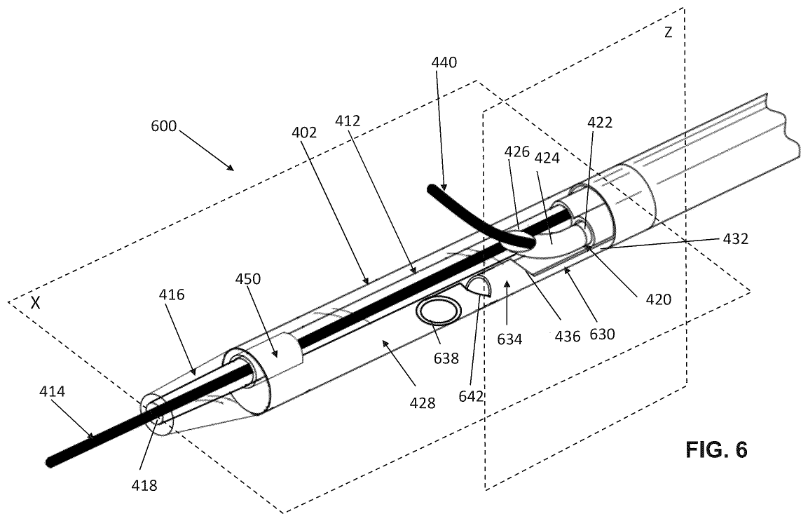

FIG. 6 depicts another variation of a reentry catheter system, comprising a catheter 600 similar in structures to the catheter 400 in FIGS. 4A and 4B, except that the ramp marker 630 comprises a longer distal segment 634 and comprises a distal end 638 that has an angled taped end. Also, an additional middle notch 642 is located between the angled segment 436 and the distal end 638. The middle notch 642 and the angled distal end 638 have different rotational orientations than the angled segment 436. In some variations, the middle notch 642 is rotated 45 degrees relative to the angled segment 436, and the angled distal end 638 is rotated 90 degrees from the middle notch 642 and 135 degrees from the angled segment 436. These two structural differences permit the user to identify additional rotational orientations of the catheter 600, compared to catheter 400 in FIGS. 4A and 4B.

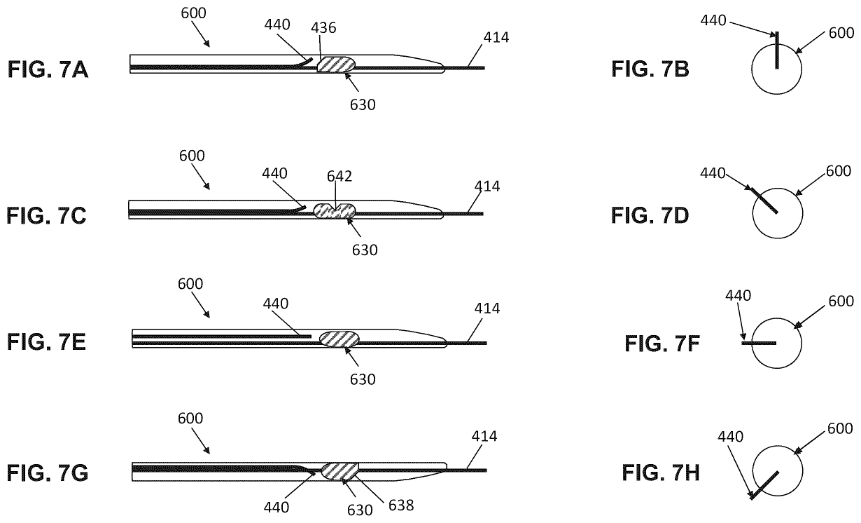

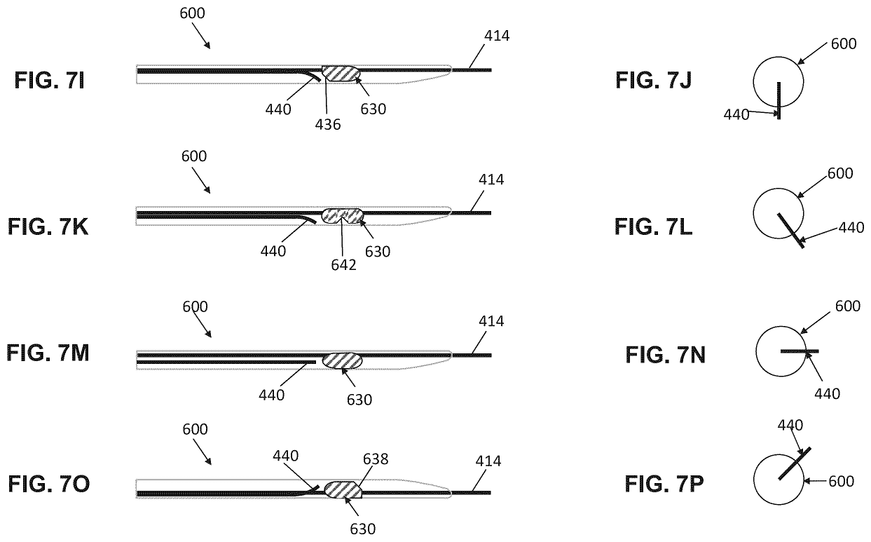

FIGS. 7A and 7B, for example, depict the catheter 600 in a “single” guidewire orientation with the positioning guidewire 414 and the reentry guidewire 440 in an overlapping or aligned configuration. In FIG. 7A, the ramp marker 630 has an angled segment 436 that is visible and extending in the cranial direction from the guidewires 414, 440, which indicates that the catheter 600 is oriented so that the reentry guidewire 440 will extend cranially (FIG. 7B).

FIGS. 7C and 7D depict the catheter 600 in an “intermediate” guidewire orientation, with the positioning guidewire 414 and the reentry guidewire 440 only partially overlapping or aligned. In FIG. 7C, however, the ramp marker 630 has a middle notch 642 that is visible and facing the cranial direction, which indicates that the catheter 600 is oriented 45 degrees between the cranial direction and the direction toward the image intensifier (FIG. 7D).

In FIGS. 7E and 7F, the catheter 600 is in a “double” guidewire configuration, with a clear separation between the positioning and reentry guidewires 414, 440. This is confirmed by the lack of any visible angled edge or middle notch on the ramp marker 630. Further, the reentry guidewire 440 is superior or cranial to the positioning guidewire 414, indicating that the catheter 600 is oriented toward the image intensifier of the fluoroscopy system, as depicted in FIG. 7F.

FIGS. 7G and 7H depict the catheter 600 in an “intermediate” guidewire orientation with the positioning guidewire 414 and the reentry guidewire 440 only partially overlapping or aligned, or minimally separated. In FIG. 7G, however, the ramp marker 630 has an angled distal end 638 visible and facing the caudal direction, which indicates that the catheter 600 is oriented 45 degrees between the caudal direction and the direction toward the image intensifier (FIG. 7H).

FIGS. 7I and 7J, for example, depicts the catheter in a “single” guidewire orientation with the positioning guidewire 414 and the reentry guidewire 440 in an overlapping or aligned configuration. In FIG. 7I, however, the ramp marker 630 has an angled segment 436 that is visible and extending caudally from the guidewires 414, 440, which indicates that the catheter 600 is oriented so that the reentry guidewire 440 will extend caudally (FIG. 7J).

FIGS. 7K and 7L depict the catheter 600 in an “intermediate” guidewire orientation with the positioning guidewire 414 and the reentry guidewire 440 only partially overlapping or aligned, or minimally separated. In FIG. 7K, the ramp marker 630 has a middle notch 642 that is visible and facing the caudal direction, which indicates that the catheter 600 is oriented 45 degrees between the caudal direction and the direction away the image intensifier (FIG. 7L).

In FIGS. 7M and 7N, the catheter 600 is in a “double” guidewire configuration, with a clear separation between the positioning and reentry guidewires 414, 440. This is confirmed by the lack of any visible angled edge or middle notch on the ramp marker 630. Further, the reentry guidewire 440 is inferior or caudal to the positioning guidewire 414, indicating that the catheter 600 is oriented away the image intensifier of the fluoroscopy system, as depicted in FIG. 7N.

FIGS. 7O and 7P depict the catheter 600 in an “intermediate” guidewire orientation with the positioning guidewire 414 and the reentry guidewire 440 only partially overlapping or aligned, or minimally separated. In FIG. 7O, the ramp marker 630 has an angled distal end 638 visible and facing the cranial direction, which indicates that the catheter 600 is oriented 45 degrees between the cranial direction and the direction away the image intensifier (FIG. 7H).

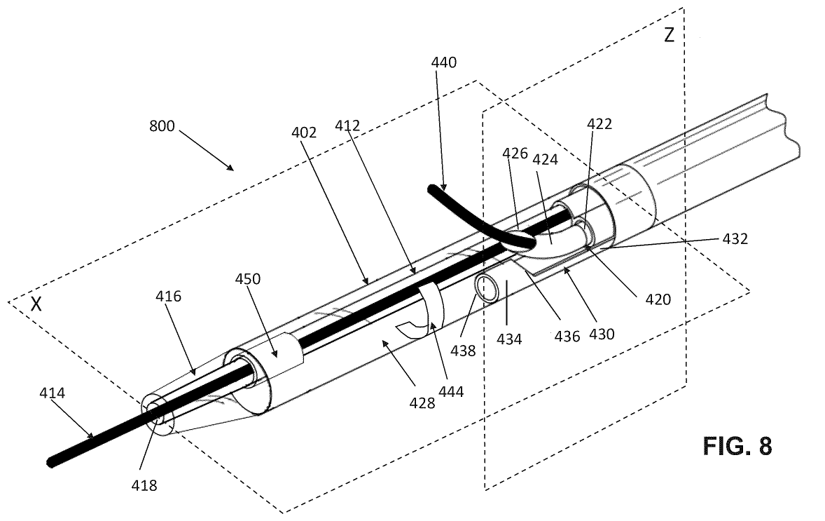

FIG. 8 depicts another variation of a reentry catheter system, comprising a catheter 800 similar in structures to the catheter 400 in FIGS. 4A and 4B, except that a partial arcuate band 444 is provided in the catheter, spaced apart distally from the distal end 438 of the ramp marker 430, and also spaced transversely away from the first through lumen 412, and has a general shape or generally corresponding to the arcuate surface of the tubular body 402, and also spaced distally away from the distal opening 426 of the distal curved segment 424 and from the ramp marker 430. This arcuate band 444 may have a hemi-circumferential configuration. In FIG. 8, the band 444 is provided at the 45 degree-135 degree-180 degree position of the catheter 800. In other variations, a different orientation may be provided at the 0 degree-90 degree-180 degree, 180 degree-270 degree-360 degree, 270 degree-360 degree-90 degree, 135 degree-190 degree-280 degree, 190 degree-280 degree-10 degree or any position between any of these two, or have a smaller circumferential configuration, for example. The addition of the band also permits determination of the catheter 800 orientation in 45 degree increments. The arcuate band 444 may be spaced least 0.5 mm, 1 mm, 1.5 mm or 2 mm, away from the ramp marker 430, to provide sufficient separation on fluoroscopy. In other variations, the separation may be in the range of 0.5 mm to 2 mm, or 0.5 mm to 1.5 mm, or between 0.5 mm to 1 mm.

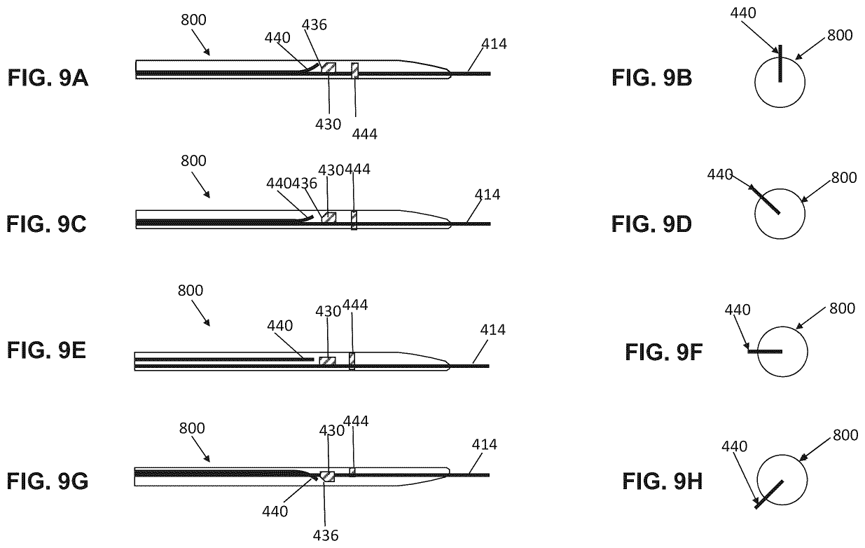

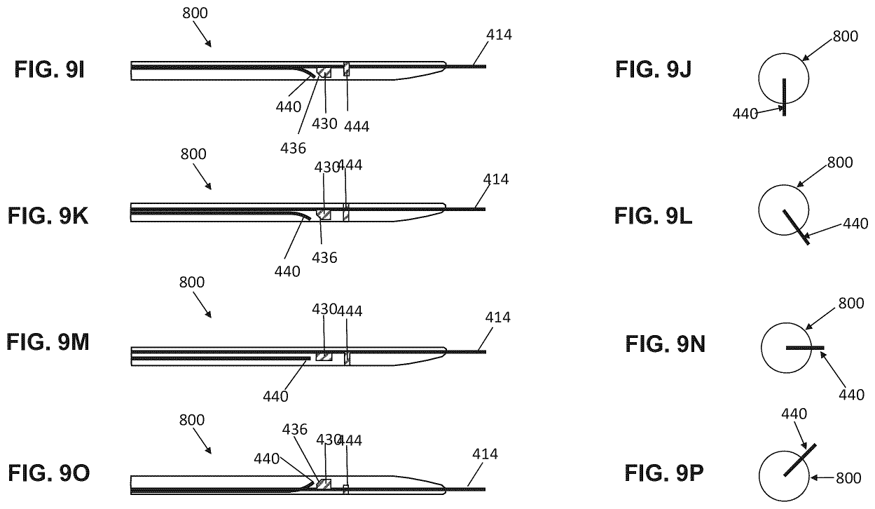

FIGS. 9A and 9B, for example, depict the catheter 800 in a “single” guidewire orientation with the positioning guidewire 414 and the reentry guidewire 440 in an overlapping or aligned configuration. In FIG. 9A, the ramp marker 630 has an angled segment 436 that is visible and extending in the cranial direction from the guidewires 414, 440. Also, the arcuate marker 444 is in an intermediate configuration, e.g. neither at maximum or minimum. This configuration indicates on fluoroscopic imaging indicates that the catheter 800 is oriented so that the reentry guidewire 440 will extend cranially (FIG. 9B).

FIGS. 9C and 9D depict the catheter 800 in an “intermediate” guidewire orientation, with the positioning guidewire 414 and the reentry guidewire 440 only partially overlapping or aligned. In FIG. 9C, however, the ramp marker 430 still has an angled segment 436 visible, and the arcuate band 444 spans the width of the catheter 800. This indicates that the catheter 800 is oriented 45 degrees between the cranial direction and the direction toward the image intensifier (FIG. 9D).

In FIGS. 9E and 9F, the catheter 800 is in a “double” guidewire configuration, with a clear separation between the positioning and reentry guidewires 414, 440. This is confirmed by the lack of any visible angled edge on the ramp marker 430. Further, the reentry guidewire 440 is superior or cranial to the positioning guidewire 414, indicating that the catheter 800 is oriented toward the image intensifier of the fluoroscopy system, as depicted in FIG. 9F. The arcuate band 444 is in an intermediate configuration.

FIGS. 9G and 9H depict the catheter 800 in an “intermediate” guidewire orientation with the positioning guidewire 414 and the reentry guidewire 440 only partially overlapping or aligned, or minimally separated. In FIG. 9G, however, the ramp marker 430 has an angled segment 436 visible and facing the caudal direction. The arcuate band 444 is at its minimum transverse dimension, which together confirms that the catheter 800 is oriented 45 degrees between the caudal direction and the direction toward the image intensifier (FIG. 9H).

FIGS. 9I and 9J, depicts the catheter in a “single” guidewire orientation with the positioning guidewire 414 and the reentry guidewire 440 in an overlapping or aligned configuration. In FIG. 9I, the ramp marker 430 has an angled segment 436 that is visible and extending caudally from the guidewires 414, 440. The arcuate band 444 is in an intermediate state, which together indicates that the catheter 800 is oriented so that the reentry guidewire 440 will extend caudally (FIG. 9J).

FIGS. 9K and 9L depict the catheter 800 in an “intermediate” guidewire orientation with the positioning guidewire 414 and the reentry guidewire 440 only partially overlapping or aligned, or minimally separated. In FIG. 9K, the ramp marker 430 that is visible and facing the caudal direction, and the arcuate band 444 is at its maximum transverse direction, which indicates that the catheter 800 is oriented 45 degrees between the caudal direction and the direction away the image intensifier (FIG. 9L).

In FIGS. 9M and 9N, the catheter 800 is in a “double” guidewire configuration, with a clear separation between the positioning and reentry guidewires 414, 440. This is confirmed by the lack of any visible angled edge on the ramp marker 430. Further, the reentry guidewire 440 is inferior or caudal to the positioning guidewire 414, indicating that the catheter 800 is oriented away the image intensifier of the fluoroscopy system, as depicted in FIG. 9N. The arcuate band 444 is also oriented in an intermediate transverse dimension.

FIGS. 9O and 9P depict the catheter 800 in an “intermediate” guidewire orientation with the positioning guidewire 414 and the reentry guidewire 440 only partially overlapping or aligned, or minimally separated. In FIG. 9O, the ramp marker 430 has an angled segment 436 visible and facing the cranial direction. The arcuate band 444 is also in a minimum transverse dimension configuration, which indicates that the catheter 800 is oriented 45 degrees between the cranial direction and the direction away from the image intensifier (FIG. 9H).

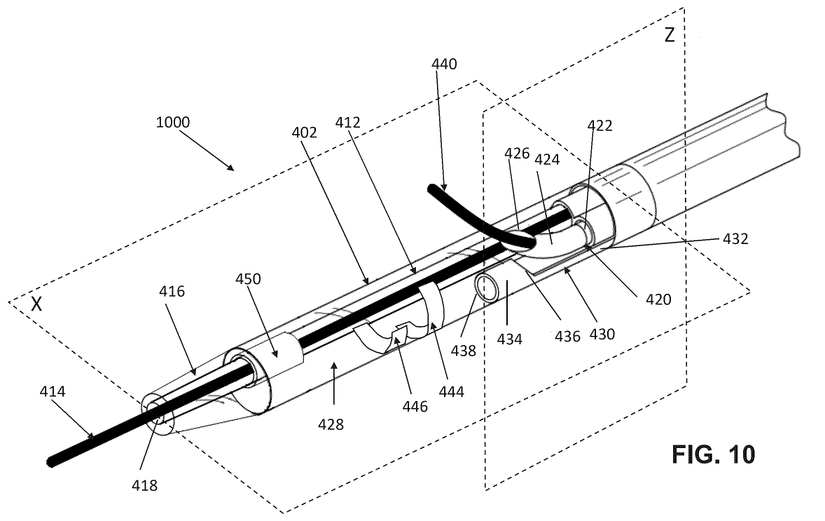

FIG. 10 depicts another variation of a reentry catheter system, comprising a catheter 1000 similar in structures to the catheter 800 in FIG. 8. One difference, however, is that a second partial arcuate band 446 is provided in the catheter, spaced apart distally from the first arcuate band 444, which in turn is spaced distally apart from the distal end 438 of the ramp marker 430. Like the first arcuate band 444, the second arcuate band 446 is spaced transversely away from the first through lumen 412 and also has a configuration or an arcuate shape that generally corresponds to the arcuate surface of the tubular body 402, and also spaced distally away from the distal opening 426 of the distal curved segment 424 and from the ramp marker 430. This second arcuate band 446 may also have a hemi-circumferential configuration, but has an orientation that is preferably 90 degrees rotated from the configuration of the first band 444. As noted previously, the first arcuate band 444 may be provided at the 45 degree-135 degree-190 degree position of the catheter 1000, but in other variations may be provided at the 0 degree-90 degree-180 degree, 180 degree-270 degree-360 degree, 270 degree-360 degree-90 degree, 135 degree-190 degree-2800 degree, 190 degree-280 degree-10 degree or any position between any of these two, or have a smaller circumferential configuration, for example. The second arcuate band 446 would be configured with an orientation that is preferably but not required to be 90 rotated relative to the first band 444.

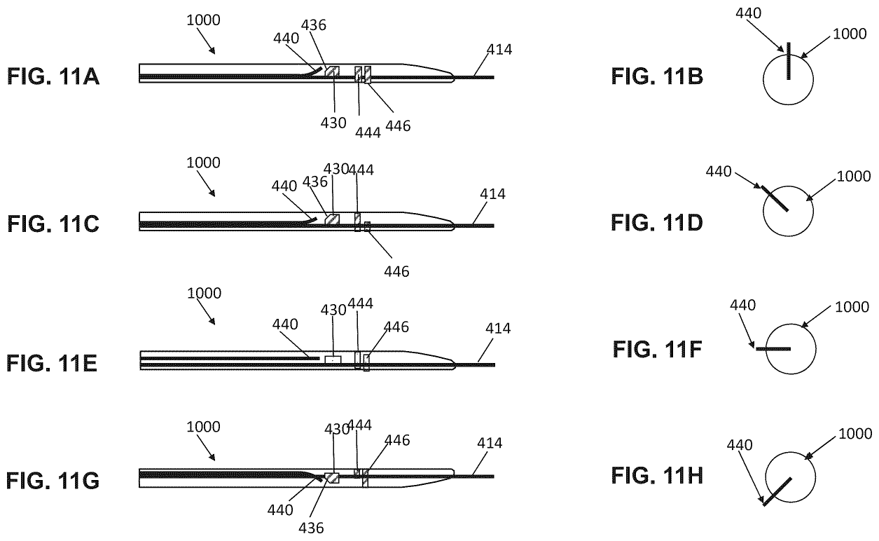

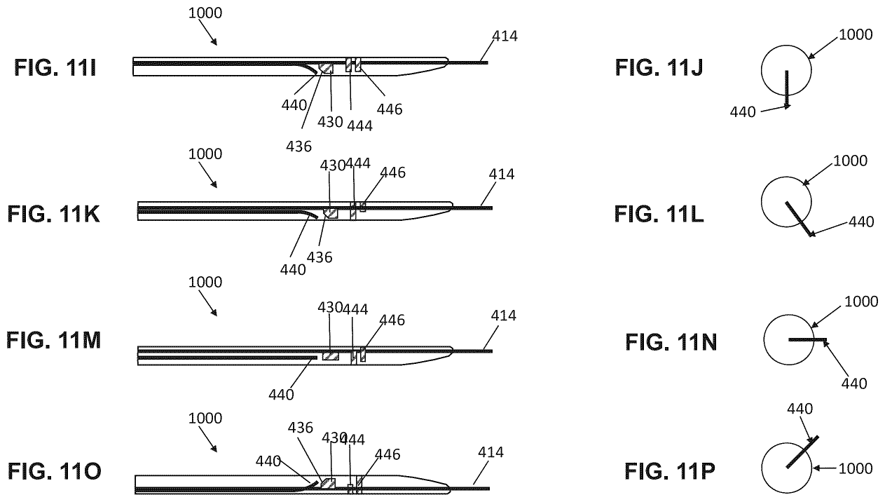

FIGS. 11A and 11B, for example, depict the catheter 1000 in a “single” guidewire orientation with the positioning guidewire 414 and the reentry guidewire 440 in an overlapping or aligned configuration. In FIG. 11A, the ramp marker 430 has an angled segment 436 that is visible and extending in the cranial direction from the guidewires 414, 440. Also, both of the arcuate markers 444, 446 are in an intermediate configuration, e.g. neither is at maximum or minimum. This configuration indicates on fluoroscopic imaging indicates that the catheter 1000 is oriented so that the reentry guidewire 440 will extend cranially (FIG. 11B).

FIGS. 11C and 11D depict the catheter 1000 in an “intermediate” guidewire orientation, with the positioning guidewire 414 and the reentry guidewire 440 only partially overlapping or aligned. In FIG. 11C, however, the ramp marker 430 still has an angled segment 436 visible, and the first/proximal arcuate band 444 is at a maximum while the second/distal arcuate band 446 is at a minimum. This indicates that the catheter 1000 is oriented 45 degrees between the cranial direction and the direction toward the image intensifier (FIG. 11D).

In FIGS. 11E and 11F, the catheter 1000 is in a “double” guidewire configuration, with a clear separation between the positioning and reentry guidewires 414, 440. This is confirmed by the lack of any visible angled edge on the ramp marker 430. Further, the reentry guidewire 440 is superior or cranial to the positioning guidewire 414, indicating that the catheter 1000 is oriented toward the image intensifier of the fluoroscopy system, as depicted in FIG. 11F. Both of the arcuate markers 444, 446 are in an intermediate configuration, e.g. neither is at maximum or minimum.

FIGS. 11G and 11H depict the catheter 1000 in an “intermediate” guidewire orientation with the positioning guidewire 414 and the reentry guidewire 440 only partially overlapping or aligned, or minimally separated. In FIG. 11G, however, the ramp marker 430 has an angled segment 436 visible and facing the caudal direction. The first proximal arcuate band 444 is at its minimum transverse dimension, while the second/distal arcuate band 446 is at its maximum, which together confirms that the catheter 1000 is oriented 45 degrees between the caudal direction and the direction toward the image intensifier (FIG. 11H).

FIGS. 11I and 11J, depicts the catheter in a “single” guidewire orientation with the positioning guidewire 414 and the reentry guidewire 440 in an overlapping or aligned configuration. In FIG. 11I, the ramp marker 430 has an angled segment 436 that is visible and extending caudally from the guidewires 414, 440. Both first/proximal arcuate band 444 and the second/distal arcuate band 446 are in an intermediate state, which all together indicates that the catheter 1000 is oriented so that the reentry guidewire 440 will extend caudally (FIG. 11J).

FIGS. 11K and 11L depict the catheter 1000 in an “intermediate” guidewire orientation with the positioning guidewire 414 and the reentry guidewire 440 only partially overlapping or aligned, or minimally separated. In FIG. 11K, the ramp marker 430 that is visible and facing the caudal direction, and the first/proximal arcuate band 444 is at its maximum transverse direction, while the second/distal arcuate band 446 is at its minimum. This indicates that the catheter 1000 is oriented 45 degrees between the caudal direction and the direction away the image intensifier (FIG. 11L).

In FIGS. 11M and 11N, the catheter 1000 is in a “double” guidewire configuration, with a clear separation between the positioning and reentry guidewires 414, 440. This is confirmed by the lack of any visible angled edge on the ramp marker 430, and that both arcuate bands 444, 446 are in the intermediate position. Further, the reentry guidewire 440 is inferior or caudal to the positioning guidewire 414, indicating that the catheter 1000 is oriented away the image intensifier of the fluoroscopy system, as depicted in FIG. 11N.

FIGS. 11O and 11P depict the catheter 1000 in an “intermediate” guidewire orientation with the positioning guidewire 414 and the reentry guidewire 440 only partially overlapping or aligned, or minimally separated. In FIG. 11O, the ramp marker 430 has an angled segment 436 visible and facing the cranial direction. The first/proximal arcuate band 444 is also in a minimum transverse dimension configuration, while the second/distal arcuate band 446 is in a maximum transverse dimension configuration, which indicates that the catheter 1000 is oriented 45 degrees between the cranial direction and the direction away from the image intensifier (FIG. 11H).

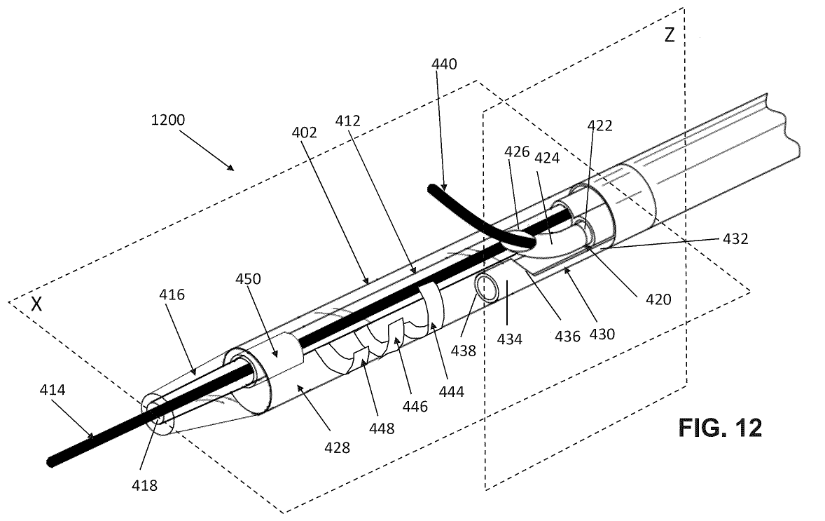

FIG. 12 depicts still another variation of a reentry catheter system, comprising a catheter 1200 similar in structures to the catheter 1000 in FIG. 10. A third partial arcuate band 448, however, is provided in the catheter 1200, spaced apart distally from the second arcuate band 446, which is spaced apart distally from the first arcuate band 444, which in turn is spaced distally apart from the distal end 438 of the ramp marker 430. This third arcuate band 448 may also have a hemi-circumferential configuration, but in contrast to catheter 1000 in FIG. 10, the three bands are have a rotational separation that is 45 degrees rather than 90 degrees apart. Like in other embodiments, the third arcuate band 448 may be spaced transversely away from the first through lumen 412, and has a configuration or arcuate shape that generally corresponds to the arcuate surface of the tubular body 402, and also spaced distally away from the distal opening 426 of the distal curved segment 424 and from the ramp marker 430As noted previously, the first arcuate band 444 may be provided at the 45 degree-135 degree-190 degree position of the catheter 1000, but in other variations may be provided at the 0 degree-90 degree-180 degree, 180 degree-270 degree-360 degree, 270 degree-360 degree-90 degree, 135 degree-190 degree-2800 degree, 190 degree-280 degree-10 degree or any position between any of these two, or have a smaller circumferential configuration, for example. The third arcuate band 448 would be configured with a rotational separation that is 135 degrees from the angled segment 436 of the distal head 434 of the ramp marker 430, and 90 degrees from the first arcuate band 444, while the second arcuate band 446 would be spaced 45 degrees from both the 1^{st} arcuate band 444 and the third arcuate band 448.

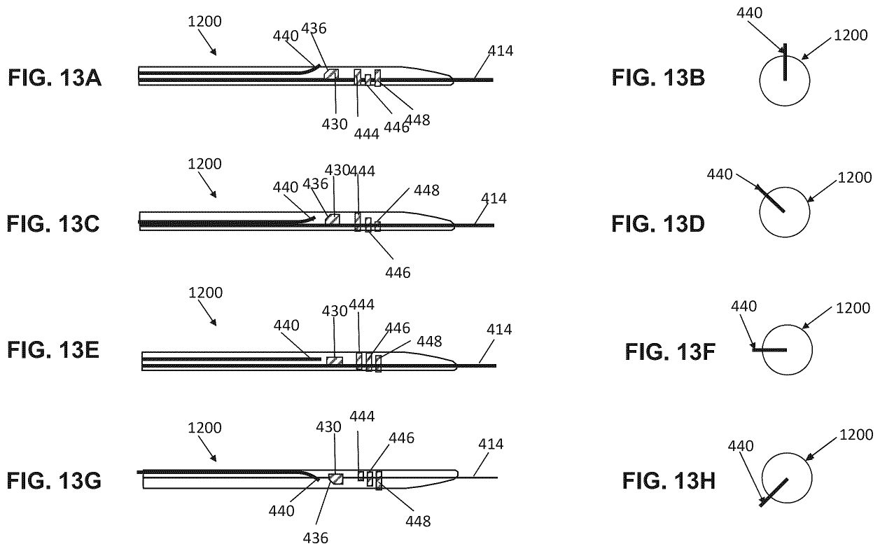

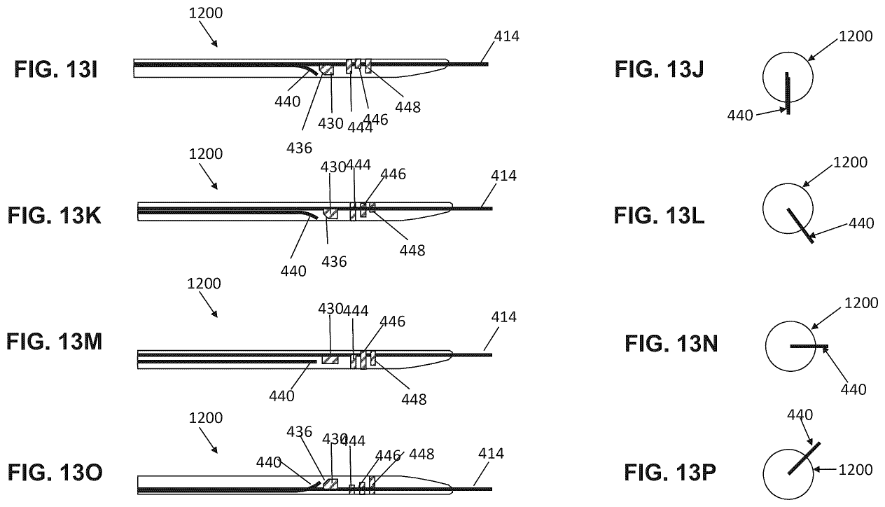

FIGS. 13A and 13B, for example, depict the catheter 1200 in a “single” guidewire orientation with the positioning guidewire 414 and the reentry guidewire 440 in an overlapping or aligned configuration. In FIG. 11A, the ramp marker 430 has an angled segment 436 that is visible and extending in the cranial direction from the guidewires 414, 440. Also, the first and third arcuate markers 444, 448 are in an intermediate configuration, while the second or middle marker 446 is at a minimum. This configuration indicates on fluoroscopic imaging indicates that the catheter 1000 is oriented so that the reentry guidewire 440 will extend cranially (FIG. 11B).

FIGS. 13C and 13D depict the catheter 1200 in an “intermediate” guidewire orientation, with the positioning guidewire 414 and the reentry guidewire 440 only partially overlapping or aligned. In FIG. 13C, however, the ramp marker 430 still has an angled segment 436 visible, the first/proximal arcuate band 444 is at a maximum, the second/middle arcuate band 4446 is intermediate while the third/distal arcuate band 448 is at a minimum. This indicates that the catheter 1000 is oriented 45 degrees between the cranial direction and the direction toward the image intensifier (FIG. 13D).

In FIGS. 13E and 13F, the catheter 1200 is in a “double” guidewire configuration, with a clear separation between the positioning and reentry guidewires 414, 440. This is confirmed by the lack of any visible angled edge on the ramp marker 430. Further, the reentry guidewire 440 is superior or cranial to the positioning guidewire 414, and both of the first and third arcuate markers 444, 448 are in an intermediate configuration, while the second/middle arcuate marker 446 is at its maximum. This configuration indicates that the catheter 1200 is oriented toward the image intensifier of the fluoroscopy system, as depicted in FIG. 13F.

FIGS. 13G and 13H depict the catheter 1200 in an “intermediate” guidewire orientation with the positioning guidewire 414 and the reentry guidewire 440 only partially overlapping or aligned, or minimally separated. In FIG. 13G, however, the ramp marker 430 has an angled segment 436 visible and facing the caudal direction. The first proximal arcuate band 444 is at its minimum transverse dimension, while the second/middle arcuate band 446 is intermediate, and the third/distal arcuate band 448 is at its maximum, which together confirms that the catheter 1200 is oriented 45 degrees between the caudal direction and the direction toward the image intensifier (FIG. 13H).

FIGS. 13I and 13J, depicts the catheter in a “single” guidewire orientation with the positioning guidewire 414 and the reentry guidewire 440 in an overlapping or aligned configuration. In FIG. 13I, the ramp marker 430 has an angled segment 436 that is visible and extending caudally from the guidewires 414, 440. Both first/proximal arcuate band 444 and the third/distal arcuate band 448 are in an intermediate state, while the second/middle arcuate band 446 is in a minimum state, which all together indicates that the catheter 1200 is oriented so that the reentry guidewire 440 will extend caudally (FIG. 13J).

FIGS. 13K and 13L depict the catheter 1200 in an “intermediate” guidewire orientation with the positioning guidewire 414 and the reentry guidewire 440 only partially overlapping or aligned, or minimally separated. In FIG. 13K, the ramp marker 430 that is visible and facing the caudal direction. The first/proximal arcuate band 444 is at its maximum transverse direction, the second/middle arcuate band 446 is intermediate at its minimum, and the third/distal arcuate band 448 is at its minimum. This indicates that the catheter 1200 is oriented 45 degrees between the caudal direction and the direction away the image intensifier (FIG. 13L).

In FIGS. 13M and 13N, the catheter 1200 is in a “double” guidewire configuration, with a clear separation between the positioning and reentry guidewires 414, 440. This is confirmed by the lack of any visible angled edge on the ramp marker 430, and that the first and third arcuate bands 444, 448 are in the intermediate position, while the second/middle arcuate band 446 is at its maximum. Further, the reentry guidewire 440 is inferior or caudal to the positioning guidewire 414, indicating that the catheter 1200 is oriented away the image intensifier of the fluoroscopy system, as depicted in FIG. 13N.

FIGS. 13O and 13P depict the catheter 1200 in an “intermediate” guidewire orientation with the positioning guidewire 414 and the reentry guidewire 440 only partially overlapping or aligned, or minimally separated. In FIG. 13O, the ramp marker 430 has an angled segment 436 visible and facing the cranial direction. The first/proximal arcuate band 444 is also in a minimum transverse dimension configuration, the second/middle arcuate band 446 is in an intermediate configuration, and the third/distal arcuate band 448 is at its maximum, which indicates that the catheter 1000 is oriented 45 degrees between the cranial direction and the direction away the image intensifier (FIG. 13H).

In other variations, a different number of orientation markers may be provided. Depending on the number of markers, the rotational spacing of the markers may vary. In some variations, for example, with N number of orientation markers, the rotational spacing of the markers would be 90/(N−1) and the number of rotational states nominally defined by the markers would be (4*N)−4. Based on these relationships the rotational spacing and number of nominal states would be:

| Number of | Degree | Number of |
|---|---|---|
| Markers | Separation | Rotational States |
| 3 | 45 | 8 |
| 4 | 30 | 12 |
| 5 | 22.5 | 16 |
| 6 | 18 | 20 |
| 7 | 15 | 24 |

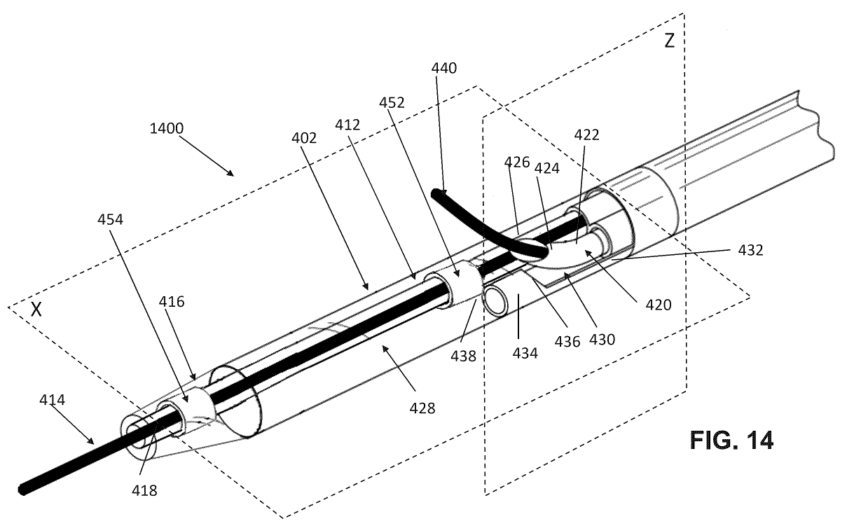

FIG. 14 depicts another variation of a reentry catheter system, comprising a catheter 1400 similar in structures to the catheter 400 in FIGS. 4A and 4B, except that a first radiopaque marker band 452 around a location of the first through lumen 412. The first marker band 452 may comprise a tubular structure, e.g. a platinum-iridium tubular structure, or may comprise a tubular coating, e.g. barium sulfate, and may have a length that is greater than the external diameter of the first guidewire lumen 412, e.g. at least 0.80 mm, 1 mm, 1.2 mm, or 1.4 mm. the proximal end of the band 452 may be aligned with the distal end of the ramp marker 430, which can assist the user with achieving the desired orientation, where the transverse dimension of the first band 452 is rotated into alignment with the transverse dimension of the ramp marker 430, so that the second ramp lumen 420 is either aligned with either the orthogonal superior or inferior direction, as further described below. In some further embodiments, a second tubular band marker 454 may be provided distal to the first marker band 452. In the specific example depicted in FIG. 14, the second tubular band 454 is may comprise a similar or same dimension as the first band 452, but may be located in the nosecone 416, or at least spaced distally from the first band 452, by at least or at 1 mm, 1.5 mm, 2 mm, 2.5 mm, 3 mm. 3.5 mm, or 4 mm, for example within the distal region of the catheter 400.

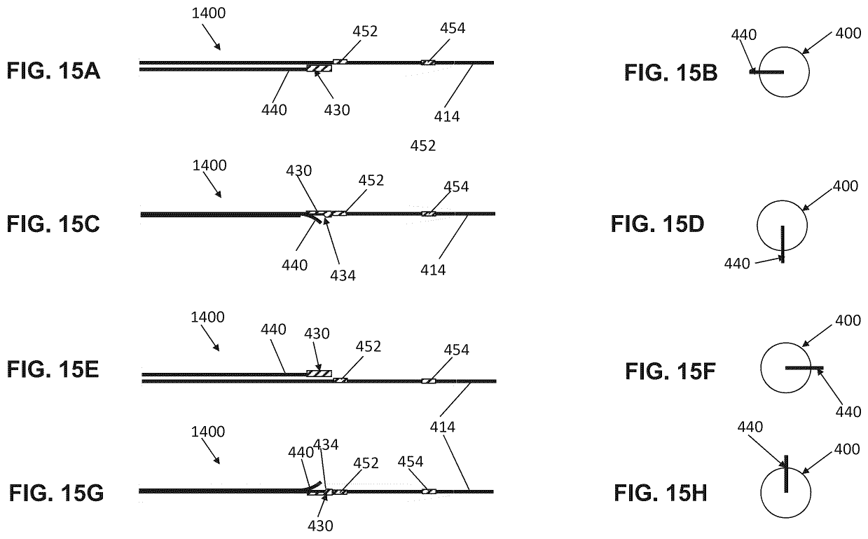

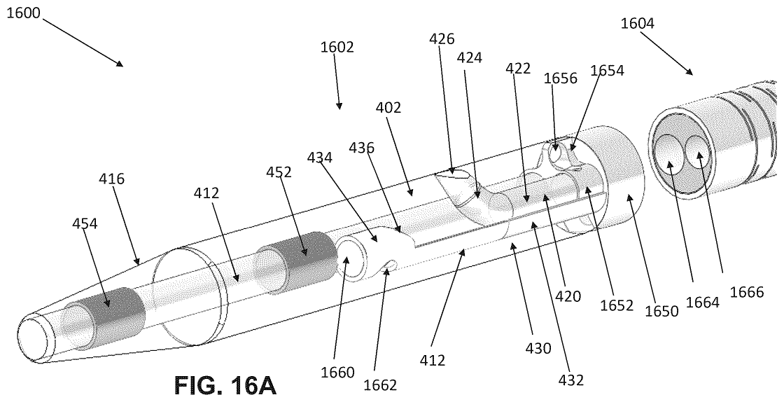

Based on this arrangement of the longitudinal alignment of the ramp marker 430 with the second ramp lumen 420 and the longitudinal alignment of the marker bands 452, 454 with the first guidewire lumen 412, and the rotational alignment of the angled segment 436 in the head or distal segment 436 of the ramp marker 430 with the orientation of the distal curved segment 424 of the ramp lumen 420, When the marker bands 452, 454 are rotationally aligned with the ramp marker 430, the visibility and orientation of the angled segment 434 will correspond with the orientation of the distal curved segment 424, in the caudal or cranial direction. When the marker bands 452, 454 are out of alignment with the ramp marker 430, the angled segment 434 will not be visible and the distal curved segment 424 is oriented either toward or away from the fluoroscopy imager, with a cranial or superior position of the marker bands 452, 454 relative to the ramp marker 430 indicative of the distal curved segment 424 oriented toward the imager and the when in the relative caudal or inferior position, indicative of the distal curved segment 424 being oriented away from the imager. These orientations are schematically depicted in FIGS. 15A to 15H.

In FIGS. 15A and 15B, the catheter 1400 is in a “double” guidewire configuration, with a clear separation between the positioning and reentry guidewires 414, 440. This is confirmed by the lack of any visible angled edge on the ramp marker 430 on fluoroscopy. Further, the reentry guidewire 440, and the two marker bands 452, 454 are superior or cranial to the positioning guidewire 414, indicating that the catheter 1400 is oriented toward the image intensifier of the fluoroscopy system, as depicted in FIG. 15B.

In FIGS. 15C and 15D, the catheter 1400 is schematically depicted in a “single” guidewire orientation with the positioning guidewire 414 and the reentry guidewire 440 in an overlapping or aligned configuration. In addition, as illustrated in FIG. 15C, the ramp marker 430 has an angled segment 434 that is visible and facing caudally from the guidewires 414, 440, which indicates that the catheter 1400 is oriented so that the reentry guidewire 440 will extend caudally (FIG. 15D).

In FIGS. 15E and 15F, the catheter 1400 is in a “double” guidewire configuration, with a clear separation between the positioning and reentry guidewires 414, 440. This is confirmed by the lack of angled segment 434 on the ramp marker 430. Further, the reentry guidewire 440 and the two marker bands 452, 454 are not aligned with the ramp marker 430, with bands 452, 454 positioned inferior or caudal to the ramp marker 430 on fluoroscopy, indicating that the catheter 1400 is oriented away the image intensifier of the fluoroscopy system, as depicted in FIG. 15F.

FIGS. 15G and 15G depict the catheter 1400 in a “single” guidewire orientation with the positioning guidewire 414 and the reentry guidewire 440 in an overlapping or aligned configuration. In FIG. 15A, the ramp marker 430 has an angled segment 434 that is visible and extending in the cranial direction from the guidewires 414, 440, which indicates that the catheter 600 is oriented so that the reentry guidewire 440 will extend cranially (FIG. 7B). This is in addition to the alignment of the ramp 430 with the marker bands 542, 454.

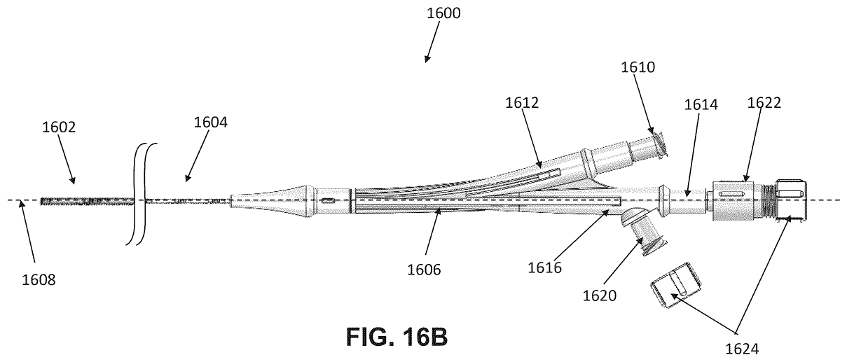

The manufacture of the various reentry catheters described herein may be performed with any of a variety of known techniques, including polymer extrusion, injection molding and/or and overmolding. Due to the number of separate components that are assembled to form the distal end of the catheters described herein, the lumens and marker components of the catheters may be pre-assembled and then attached to the proximal end of the catheter. In FIG. 16A, for example, the catheter 1600 comprises a distal segment 1602 that is attached to an elongate multi-lumen catheter body 1604, which in turn is attached to a multi-port hub 1606, depicted in FIG. 16B. The hub 1606 comprises a longitudinal axis 1608, with angled or curved port 1610 with a curved or angled conduit body 1612 that is in fluid communication with the ramp lumens of the catheters described herein, and a straight port 1614 that is aligned with or parallel to the longitudinal axis 1608 of the hub 1606, with a straight conduit body 1616, which is in fluid communication with the guidewire lumens of the catheters described herein. Secondary ports 1620 may also be optionally provided on the curved conduit body 1612 and/or the straight conduit body 1616, to provide additional access and/or flush functionality to the guidewire or ramp lumens, when a guidewire is already inserted into the main ports 1610 and 1614. Each port may comprise a standard connector configuration, e.g. Luer, with a hemostasis valve 1622 provided either in the port and/or a port cap 1624.

Referring to back to FIG. 16A, which depicts the distal segment 1602 of the catheter 1600 separate from the multi-lumen catheter body 1604, the distal segment 1602 may comprise a base body 1650 with multiple lumens or recesses which are aligned with or configured to receive the tubular bodies of the first guidewire lumen 412 and the ramp lumen 420, as well the elongate tail or proximal segment 432 of the ramp marker 430, that is aligned with or abutting the proximal straight segment 422 of the ramp lumen 420, while also spacing apart the enlarged head or distal segment 434 of the ramp marker 430 which provides the angled surface or segment 436 used to achieve the desired catheter orientation. The base body 1650 may comprise one or more tubular extensions 1652 to provide greater lumen support and/or attachment surface between the base body 1650 and the lumens 412, 420. One or more base flanges 1654 that extend from the tubular extensions 1652 or the base body 1650 to increase the surface area contact when the body 1650 of the distal segment 1602 is molded over all of the subassembly components. The flange 1654 may further comprise an opening 1656, which may allow polymer flow-through during the molding process to further resist polymer separation from the subassembly components during use. Likewise, while the ramp marker 430 may comprise an enlarged head 434 or distal segment that comprises a tubular configuration with a lumen 1660 that also permits polymer flowthrough. Additional transverse or side openings 1662 may be provided in the proximal or distal segments 432, 434 of the ramp marker 430 to provide additional polymer inflow during manufacturing. The nosecone 416 and tubular body 402 of the distal segment 1602 are then formed during the molding process and then the distal segment 1602 can then be welded, adhered or otherwise attached to the catheter body 1604. Mandrels (not shown) may be inserted into the distal segment 1602 and/or catheter body 1604 to assist with the alignment of the catheter body lumens 1664, 1666 that correspond to the first guidewire lumen 412 and the second ramp lumen 420, respectively.

The catheters describes herein may be used in conjunction with steerable guidewires in order to access discrete regions of the coronary and peripheral arterial vasculature, to facilitate placement and exchange of guidewires and other interventional devices, for use during two guidewire procedures and to subselectively infuse/deliver diagnostic or therapeutic agents. In other embodiments, they may be used with other vasculature, such as the venous vasculature, and other body lumens, such as lymph channels and the gastrointestinal tract, biliary tree, etc. In yet another embodiment of the catheters, with a smaller form factor and smaller accompanying guidewires notwithstanding, may be used for neurovasculature procedures.

In one embodiment, a method of using a catheter as described herein includes prepping and draping the patient in usual sterile fashion and achieving anesthesia as needed for a cardiac catheterization and that a tracking guidewire has already been positioned at the target location.
1. In addition to the catheter package, a hemostasis valve (e.g. Tuohy Borst type), guidewires, a syringe (e.g. 10 mL for flushing) and sterile heparinized saline for flushing is also provided.
2. Inspect the catheter pouch for damage. Do not use the catheter if the package has been opened or damaged.
3. Using sterile technique, remove the catheter from the package and transfer to the sterile field. Inspect the catheter for any visible signs of damage.
4. Hydrate the catheter tip in saline for 30 seconds.
5. Flush the catheter with heparinized saline with a 5 or 10 cc syringe through the positioning lumen flush port (e.g. port 1620 in FIG. 16B) and verify that fluid comes out of the distal tip of the catheter.
6. Flush the catheter with heparinized saline through the tracking guidewire flush port with a 5 or 10 cc syringe and verify that fluid comes out of the distal tip (e.g. port 1614 in FIG. 16B).
7. Backload the catheter onto the tracking guidewire that is already in place in the vasculature.
8. Carefully advance the catheter, under visual (e.g., fluoroscopy) guidance, over the tracking guidewire and through the guide catheter until the catheter tip is just proximal to the end of the guide catheter. Observe the tip of the catheter under fluoroscopy as the operator advances the catheter out of the guiding catheter and into the vasculature. Stop advancing the catheter when the catheter tip has reached the targeted lesion site.
9. The catheter should not be advanced or withdrawn against resistance until the cause of resistance is determined by fluoroscopy. Movement of the catheter against resistance may result in catheter damage or vessel injury.
10. To deliver the second wire through the positioning port (e.g. port 1610 in FIG. 16B), load the positioning guidewire into the introducer positioning port of the catheter and advance under fluoroscopy until the distal tip of the guidewire reaches the distal nosecone of the catheter. The guidewire should remain within the nosecone until the catheter is aimed into the final desired direction.
11. Use the guidewire introducer (e.g. catheter hub 1606) to rotate the catheter and aim or orientate the catheter in the intended direction. As the operator rotates or orientates the catheter, x-ray fluoroscopy displays realtime shadow projection of the markers at the distal end of the catheter (nosecone) providing orientational information as to the direction of the positioning guidewire exiting the side port, as schematically depicted in FIGS. 15A, 15C, 15E and 15G.
12. Once the operator has made the decision to deploy the guidewire in the desired direction, advance the guidewire out the ramp toward and into the desired location.
13. Typically, once the site and direction of guidewire positioning/re-entry is determined, the operator may also replace the existing guidewire via the fuidewire introducer. The operator should make the independent decision should a guidewire of different type or stiffness is more appropriate.
14. If guidewire is stuck in the catheter during advancement or retraction, the guidewire lumen may be flushed through the guidewire lumen flush port or positioning guidewire lumen with heparinized saline to help free the guidewire within the guidewire lumen.
15. When pulling guidewire back into catheter, the user should stop if resistance is felt and determine the cause of the resistance. If guidewire cannot be freed up with flushing heparinized saline through the guidewire lumen flush port or positioning guidewire lumen or if the guidewire prolapses or kinks, do not pull guidewire back into the catheter; slowly and gently under fluoroscopic guidance remove the guidewire and catheter as a unit.
16. The catheter may then be used to perform a vascular reentry procedure to bypass a luminal lesion in the vasculature by transiting from a pre-lesional lumen location in the vasculature into the vessel wall, along the vascular wall and back into the lumen at a post-lesion lumen location.
17. When the procedure is completed after proper placement of the guidewire, carefully retract catheter while leaving the guidewire in place using standard catheterization lab procedures.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean±10%, ±5%, or +2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. A catheter, comprising:
a tubular catheter body, the tubular catheter body comprising:
a first lumen comprising a first proximal opening, a first distal opening and a first straight lumen therebetween;
a second lumen comprising second proximal opening, a second distal opening, a second straight lumen and a curved lumen, wherein the second distal opening is proximal to the first distal opening and located in a sidewall of the tubular catheter body, and wherein the curved lumen is located between the second straight lumen and the second distal opening;
a radiopaque angled ramp structure;
wherein the first straight lumen and second straight lumen are parallel and located in a first plane;
wherein the second straight lumen and the curved lumen are located in a second plane that is non-parallel to the first plane;
wherein the radiopaque angled ramp structure extends from the first plane and is in alignment with the second plane.

2. The catheter of claim 1, wherein the radiopaque angled ramp structure comprises an obtuse angled surface relative to the second straight lumen of the second lumen, the obtuse angled surface lying in a third plane.

3. The catheter of claim 2, wherein the radiopaque angled ramp structure further comprises a distal end with an orthogonal configuration.

4. The catheter of claim 2, wherein the radiopaque angled ramp structure further comprises a distal end with a 30 to 60 degree angled surface lying in a fourth plane.

5. The catheter of claim 4, wherein the radiopaque angled ramp structure further comprises a notch located between the obtuse angled surface and the distal end.

6. The catheter of claim 5, wherein the notch comprises an angled notch surface located in a fifth plane that is different from the third plane.

7. The catheter of claim 5, wherein the fifth plane is axially rotated 45 degrees from the third plane.

8. The catheter of claim 7, wherein the fourth plane is axially rotated 45 degrees from the fifth plane.

9. The catheter of claim 8, wherein the fourth plane is axially rotated 90 degrees from the third plane.

10. The catheter of claim 3, further comprising a first circumferentially oriented arcuate radiopaque band spaced distally from the distal end of the ramp marker.

11. The catheter of claim 10, wherein the first circumferentially oriented arcuate radiopaque band is a hemi-circumferential band.

12. The catheter of claim 10, further comprising a second circumferentially oriented arcuate radiopaque band spaced distally from the first circumferentially oriented arcuate radiopaque band, the second circumferentially oriented arcuate radiopaque band comprising a different rotational orientation than the first circumferentially oriented arcuate radiopaque band.

13. The catheter of claim 12, wherein the second circumferentially oriented arcuate radiopaque band is a hemi-circumferential band.

14. The catheter of claim 13, wherein the second circumferentially second circumferentially oriented arcuate radiopaque band is rotated 90 degrees from the first circumferentially oriented arcuate radiopaque band.

15. The catheter of claim 3, further comprising a tubular radiopaque marker surrounding the first lumen and longitudinally aligned with the orthogonal configuration of the distal end of the ramp structure.

16. The catheter of claim 1, wherein the angle between the first plane and second plane is in the range of 60 to 80 degrees.

17. The catheter of claim 1, further comprising a base body coupled to the first lumen, the second lumen, and the angled ramp structure.

18. The catheter of claim 17, further comprising a base body flange extending from the base body, the flange comprising a radial longitudinal orientation and having a through hole.

19. The catheter of claim 1, wherein the angled ramp structures comprises an elongate proximal tail, and an enlarged distal head, wherein the enlarged distal head comprises a tubular shape and a transverse opening.

20. (canceled)

21. (canceled)

22. The catheter of claim 1, wherein the second plane is orthogonal or forms an acute angle to the first plane.
